(19) United States
(12) Patent Application Publication
(10) Pub. No.: US 2025/0222002 A1
(43) Pub. Date: Jul. 10, 2025

(54) METHODS FOR TREATING BENZODIAZEPINE MISUSE/USE DISORDER

(71) Applicant: University of Mississippi Medical Center, Jackson, MS (US)

(72) Inventors: James K. Rowlett, Ridgeland, MS (US); David James Pearson, Nottingham (GB); Farhan Kayyum Taherali, Nottingham (GB); Huong Thi Thanh Nguyen, Nottingham (GB)

(21) Appl. No.: 19/056,599

(22) Filed: Feb. 18, 2025

Related U.S. Application Data
(63) Continuation-in-part of application No. 17/361,754, filed on Jun. 29, 2021, now Pat. No. 12,233,070.
(60) Provisional application No. 63/046,615, filed on Jun. 30, 2020.

(51) Int. Cl. A61K 31/519; A61P 25/30; C07C 57/15; C07D 487/04

(52) U.S. Cl. CPC A61K 31/519; A61P 25/30; C07C 57/15; C07D 487/04

(57) ABSTRACT

Disclosed are compositions and methods for treating benzodiazepine misuse and/or use disorder. A method of treating benzodiazepine misuse and/or use disorder includes administering to a subject in need thereof of an effective amount of a compound that provides both partial modulator and antagonist effects at GABA_{A} receptors.

### CROSS-REFERENCE TO RELATED APPLICATIONS

This application is a continuation-in-part of U.S. patent application Ser. No. 17/361,754, filed Jun. 29, 2021, which claims the benefit of and priority to U.S. Provisional Patent Application No. 63/046,615, filed Jun. 30, 2020. Each of the foregoing applications is incorporated herein by this reference.

### STATEMENT REGARDING FEDERALLY SPONSORED RESEARCH

This invention was made with government support under R01 DA011792 awarded by the National Institutes of Health. The government has certain rights in the invention.

### BACKGROUND

### Technical Field

This disclosure relates to compositions for use in treating benzodiazepine misuse and/or use disorder and related methods of using such compounds for treating benzodiazepine misuse and/or use disorder.

### Related Technology

Benzodiazepines (also referred to as “BZs”) were originally developed as anxiety-reducing (“anxiolytic”) and sedative drugs for treating anxiety and sleep disorders. Commonly prescribed benzodiazepines include diazepam (trade name: Valium®), alprazolam (trade name: Xanax®), clonazepam (trade name: Klonopin®), lorazepam (trade name: Ativan®), midazolam (trade name: Versed®), and temazepam (trade name: Restoril®). The core of a benzodiazepine is a fusion of a benzene ring and a diazepine ring, with a sometimes-substituted phenyl group attached to the diazepine.

Since their introduction in the 1960's, benzodiazepines have been associated with liability for abuse and dependence. Benzodiazepine abuse can be characterized as “misuse” (use outside of prescription directions) and “use disorder” (as defined by WHO guidelines, a mental disorder in which benzodiazepine consumption is uncontrolled and detrimental to psychosocial functioning, usually accompanied by physical dependence).

Recent epidemiological findings suggest that the abuse of benzodiazepines may be on the rise, with misuse among U.S. adults accounting for nearly 20% of substance use overall. Benzodiazepine prescription rates remain relatively high in the U.S. (˜8% of the population), with females prescribed at rates approximately 3-fold higher than men across all age groups. U.S. treatment admissions due to benzodiazepine use increased 43% from 2006 to 2016. Of particular concern is the increase in overdose deaths caused by benzodiazepines combined with opioids. Moreover, in concert with the COVID-19 pandemic, anti-anxiety prescriptions in the U.S. increased 34.1% from mid-February to mid-March in 2020.

Despite these issues, no approved and broadly effective pharmacotherapy exists for the treatment of benzodiazepine misuse and/or use disorder. Current options are off-label use of other benzodiazepines, gabapentin (a drug used for neuralgia treatment), or sodium phenobarbital (an anesthetic barbiturate). These treatment options have minimal supportive evidence of efficacy in treating benzodiazepine misuse and/or use disorder. Moreover, use of barbiturates such as sodium phenobarbital is concerning given the toxicity and abuse liability of such drugs.

Accordingly, there is an ongoing need for improved therapies for treating benzodiazepine misuse and/or use disorder. Such therapies should ideally be capable of effectively suppressing the effects of conventional benzodiazepines without themselves introducing increased abuse potential. Such therapies should also ideally provide such effects without introducing significant motor coordination deficits, appetite suppression, respiratory suppression, or other unwanted physiological side effects. Such therapies should also ideally provide such effects without precipitating benzodiazepine withdrawal.

### SUMMARY

This disclosure is directed to compositions and methods for treating benzodiazepine misuse and/or use disorder. Compounds disclosed herein can have mixed partial modulator and antagonist profiles. These partial modulator/antagonist compounds have reduced abuse potential relative to benzodiazepines yet retain the ability to reduce anxiety. Compounds disclosed herein are capable of blocking the effects of benzodiazepines directly (i.e., independent of sedative or motor effects of the compound). Compounds disclosed herein can avoid precipitation of withdrawal following bolus doses of benzodiazepines, suggesting an exceptional ease-of-transition to treatment.

In one embodiment, a method of treating benzodiazepine misuse and/or use disorder comprises administering to a subject in need thereof an effective amount of a compound that provides both partial modulator and antagonist effects at GABAA receptors. The compound may be a compound of Formula I, Formula II, Formula III (as those Formulas are described in more detail below), or a combination or mixture thereof.

In some embodiments, the treatment provides an anxiolytic effect, but wherein positive reinforcing effects of the compound are less than from a conventional benzodiazepine. In some embodiments, the compound functions to suppress the effects of a conventional benzodiazepine (e.g., reduce self-administration) independent of sedative effects or motor effects of the administered partial modulator/antagonist compound. In some embodiments, the suppression is insurmountable and thus cannot be negated by increased benzodiazepine dosing.

In some embodiments, the treatment does not induce substantial motor coordination deficits in the subject, does not induce substantial appetite suppression in the subject, and/or does not substantially affect breathing frequency, tidal volume, or minute volume in the subject.

In some embodiments, the treatment does not precipitate benzodiazepine withdrawal. For example, the compound may be administered without prior or concurrent detoxification treatment of the subject, or with less detoxification as compared to conventional treatment options. In some embodiments, the treatment can not only avoid withdrawal-precipitating effects, but can reverse withdrawal effects (e.g., due to benzodiazepine misuse and/or as induced by a GABA_{A} receptor antagonist).

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIGS. 1A and 1B illustrate the GABA_{A} receptor and its protein family subunits, showing allosteric binding to GABA_{A} receptors and enhancement of the ability of GABA to increase chloride conductance.

FIGS. 2 through 4 illustrate exemplary compounds according to Formulas I, II, and III, respectively, that may be utilized to treat benzodiazepine misuse and/or use disorder.

FIG. 5 illustrates break points (highest response requirement obtained) for maximally self-administered doses of lorazepam and TPA023B (doses are in parenthesis on x-axis, in mg/kg/injection). These values were compared with break points obtained when vehicle was available. Data are median and interquartile ranges for n=4 monkeys and were analyzed with Friedman's repeated measures ANOVA on ranks. Note that *p<0.05, Dunnett's tests for ranks.

FIG. 6 illustrates self-administration of midazolam (MZ, 0.018 and 0.056 mg/kg/injection) and food (2 pellets/delivery) under a PR schedule, following TPA023B pretreatments. Data are mean number of reinforcers, +/−SEM, with a “Reinforcer” consisting of an injection of midazolam or delivery of 2 pellets. V, midazolam vehicle. N=4 monkeys.

FIGS. 7A and 7B illustrate dose-response function for midazolam self-administration in the PR procedure (N=4 monkeys). FIG. 7A: data as mean # injections/session +/−SEM. FIG. 7B: data as mean % maximum, obtained as maximal # injections/session for individual monkeys. Error bars are omitted from the bottom panel for clarity.

FIG. 8 illustrates a comparison of TPA023B anti-conflict effects and blockade of midazolam self-administration in rhesus monkeys. Data are percent maximal effects for individual monkeys, averaged together. N=8 (4 for conflict, 4 for self-administration).

FIG. 9 illustrates pulmonary ventilation following alprazolam and TPA023B administration in adult male Sprague-Dawley rats (N=8). Respiration parameters were measured via whole body plethysmography and presented as mean +/−SEM for each measure. Note that *p<0.05 vs. vehicle treatment (“Veh”), Bonferroni t-tests. TPA023B had no effects on any respiratory measure, either by ANOVA or Bonferroni t-tests.

FIGS. 10A to 10C illustrate effects of the full modulator diazepam, the antagonist flumazenil, and the selective partial modulator TPA023B on rates of responding maintained by food in rats (N=8). Data are mean +/−percent of baseline responding prior to the test sessions. Note that *p<0.05, vs. vehicle (V), Bonferroni t-tests.

FIGS. 11A and 11B illustrate effects of flumazenil and TPA023B on rates of responding following 3.0 mg/kg of diazepam administered 60 min prior to the session. V, vehicle (i.e., diazepam alone). Note that *p<0.05 vs. ligand administered alone, Bonferroni t-tests.

FIGS. 12A and 12B illustrate effects of the full modulator diazepam, the antagonist flumazenil (Flu), and the selective partial modulators L-838,417 and MRK-623 on rates of responding maintained by food in rats (N=5). Data are mean +/−percent of baseline responding prior to the test sessions. Note that *p<0.05, vs. vehicle (Veh), Bonferroni t-tests. “ns”, non-significant.

FIG. 13 illustrates results of testing on rhesus monkeys demonstrating that TPA023B lacks withdrawal-precipitating effects and can reverse withdrawal induced by the non-selective GABA_{A} antagonist flumazenil.

FIG. 14 illustrates results of testing on rhesus monkeys demonstrating that MRK-898 can suppress benzodiazepine self-administration and that suppression could not be surmounted with increased benzodiazepine dose.

FIGS. 15A-15F illustrate sedative and motor effects of MRK-898 on rhesus monkeys, showing that MRK-898 does not induce sedative or motor effects over a 100-fold dose range.

FIGS. 16A-16F illustrate that MRK-898 can block the sedative-motor effects of a standard benzodiazepine in rhesus monkeys.

FIGS. 17A and 17B illustrate that orally dosed MRK-898 can also block the sedative-motor effects of a standard benzodiazepine in rhesus monkeys.

FIGS. 18A-18D illustrate results of a prophetic example comparing a conventional GABA antagonist (flumazenil) to MRK-898 following pre-treatment with a benzodiazepine, the results showing that MRK-898 is predicted to minimize or avoid precipitation of withdrawal.

### DETAILED DESCRIPTION

### Overview of Benzodiazepines

Embodiments described herein are directed to compounds and methods for treating benzodiazepine misuse and/or use disorder. Benzodiazepines have the following general structure:

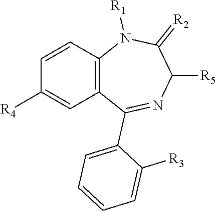

As shown, the core of a benzodiazepine is a fusion of a benzene ring and a diazepine ring, with an optionally substituted phenyl group attached to the diazepine. R₁ is typically H or CH₃; R₂ is typically O; or R₁ and R₂ together form an optionally substituted diazole or triazole ring fused to the diazepine ring. R₃ is typically Cl, F, or is absent. R₄ is typically Cl, F, or a nitro group. R₅ is typically an OH or is absent.

Other benzodiazepine-like drugs are not technically benzodiazepines but involve similar treatment targets and mechanisms of action and are therefore sometimes associated with benzodiazepines. For example, certain non-benzodiazepine hypnotics, often referred to as “Z-drugs” are structurally different from benzodiazepines but are similarly utilized to modulate the GABA_{A} receptor and are also sometimes associated with misuse and/or use disorder. The Z-drugs include zopiclone, eszopiclone (trade name: Lunesta®), zaleplon (trade name: Sonata®), and zolpidem (trade name: Ambien®).

The compositions and methods described herein may be utilized to treat misuse and/or use disorder of benzodiazepines, Z-drugs, or both. For simplicity, the term “benzodiazepine(s)” will be used to refer to both conventional benzodiazepines (i.e., those compounds having a benzodiazepine structure as illustrated above) and benzodiazepine-like drugs such as Z-drugs. The term “conventional benzodiazepine(s)” will be used to specify compounds having a benzodiazepine structure.

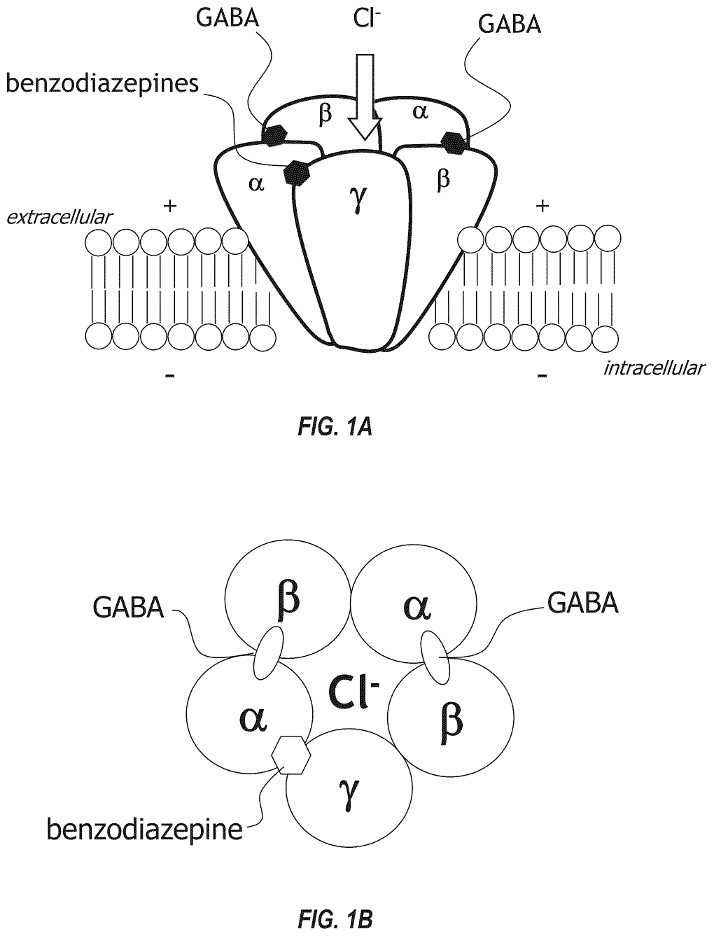

Benzodiazepines act by binding allosterically to GABA_{A} receptors and enhancing the ability of GABA to increase chloride conductance. Benzodiazepines are therefore positive allosteric modulators of GABA. As illustrated in FIGS. 1A and 1B, GABA_{A} receptors in the central nervous system are pentamers including structurally distinct proteins, with each protein family comprised of different subunits. The majority of GABA_{A} receptors consist of α, β, and γ subunit families arranged in the sequence αβαβγ as viewed from “above” the synapse (FIG. 1B).

The effects of benzodiazepines appear to be determined predominantly by the presence of particular α subunits. Benzodiazepine-type drugs bind to a site on the native GABA_{A} receptor located at the interface of the γ2 subunit with either α1, α2, α3, or α5 subunits (i.e., α1GABA_{A}, α2GABA_{A}, α3GABA_{A}, α5GABA_{A} subtypes; respectively), whereas most of these drugs are inactive at corresponding α4- and α6-subunit containing receptors (i.e., α4GABA_{A}, α6GABA_{A} subtypes; respectively). Embodiments described herein primarily modulate the GABA_{A} receptor via interactions with α1GABA_{A}, α2GABA_{A} and α3GABA_{A} receptor subtypes, which are believed to be important for mediating the abuse-related and anxiolytic effects of benzodiazepines.

### Exemplary Partial Modulator/Antagonist Compounds

Compound design and discovery in the area of positive modulators of GABA_{A} receptors have been hampered by difficulties in addressing selectivity for α2GABA_{A} and α3GABA_{A} subtypes (Atack 2011, Current Topics in Med Chem, 11:1176-202; Maramai et al. 2020, J Med Chem 63:3425-46). Little success has been achieved in identifying compounds with “selective affinity”, i.e., different binding constants (potencies) for different subtypes. Instead, the approach that has emerged is “selective efficacy”, in which intrinsic efficacy at subtypes or subtype combinations that represent targets range from full to partial modulation, whereas subtypes or subtype combinations that are associated with side effects have intrinsic efficacy ideally at zero (i.e., are antagonists at these sites). Assuming that antagonists have no functional effects, then behaviors engendered at the sites with >0 efficacy can be interpreted as mediated by those sites.

The concept of intermediate efficacy, or “partial agonism” has driven drug discovery based on the observation that lower levels of in vitro efficacy predict weaker behavioral effects. For allosteric modulators, the equivalent term is “partial modulator”. Certain embodiments described herein beneficially function as subtype-selective partial modulators and as antagonists.

Table 1 lists a series of compounds developed originally by Merck & Co. that demonstrate no differences in receptor subtype affinity but act as partial positive modulators for particular subtype combinations while having zero efficacy (i.e., antagonism) at other subtypes. The chemistry and basic pharmacology of these compounds have been reviewed in the literature (reviewed by Atack 2011). Notably, however, prior research has not explored the potential use of such compounds for treating benzodiazepine misuse and/or use disorder.

TABLE 1
Compound series with activity at α1, α2, α3, and α5
subunit-containing GABA_{A} receptors

| | | | Subtype | | | |
|---|---|---|---|---|---|---|
| Chemical Series | Compound | Pharmacokinetics*** | α1 | α2 | α3 | α5 |
| Imidazo- | TPA023B | good | none | partial | partial | partial |
| triazines | MRK-968 | good | none | partial | partial | none |
| | MRK-973 | good | none | partial | partial | partial |
| Imidazo- | MRK-623 | poor | none | partial | partial | none |
| pyrimidines | MRK-898 | good | none | partial | partial | none |
| Triazolo- | MRK-696 | poor | partial | partial | partial | partial |
| pyridazines | L-838,417* | poor | none | partial | partial | partial |
| | TPA023** | good | none | partial | partial | none |
| | MRK-409 | good | partial | partial | partial | partial |
| Pyridazines | MRK-257 | good | none | partial | partial | partial |
| | MRK-547 | good | none | partial | partial | none |
| | MRK-100 | good | none | none | partial | none |

*deuterated form developed by Concert Pharmaceuticals, out-licensed to Cipla Technologies.
**Halted in Phase II due to adverse effects (cataracts; Möhler 2011, Neuropharmacology 60: 1042-49), post hoc analysis revealed anxiolysis in patients with Generalized Anxiety Disorder.
***“poor” - low bioavailability and/or short half-life; “good” - potent with long half-life.

The imidazotriazine TPA023B was researched for its use as a non-sedating anxiolytic drug (Atack 2011). In Merck's preclinical programs, TPA023B was found to be minimally sedating (rodent rotarod and chain-pulling, squirrel monkey lever pressing) and to have anxiolytic-like effects (rodent elevated plus maze, conditioned suppression of drinking, fear-potentiated startle; squirrel monkey conditioned emotional response; Atack et al. 2010, J Psychopharmacol 25:329-344). Using both in vivo occupancy and imaging studies, minimally-effective doses of TPA023B in anxiolysis assays in rodents and primates occurred at doses occupying 61-88% of CNS benzodiazepine binding sites (Atack et al. 2010).

In a Phase I Clinical Trial (Atack et al. 2010), TPA023B was orally-dosed in men up to 3.0 mg/70 kg. This dose engendered fatigue and drowsiness in some subjects (1-4 subjects out of 8 total). At a dose of 1.5 mg/70 kg, however, no adverse effects were obtained. This dose resulted in 50-55% occupancy of CNS benzodiazepine binding sites, as measured via PET. No ataxia was observed at any dose. These mild sedative effects are consistent with findings in monkeys.

These prior investigations were focused on developing a non-sedating anxiolytic, and therefore did not discuss the investigated compounds in the context of treating benzodiazepine misuse and/or use disorder. While even a small degree of drowsiness might be considered detrimental in the development of an anxiolytic, such effects likely are acceptable for applications in benzodiazepine misuse and/or use disorder. Moreover, these mild sedative effects are significantly less than typically observed with off-label pharmacotherapies used currently to treat this disorder, e.g., phenobarbital, gabapentin, or tapering with other conventional benzodiazepines.

Other unwanted side effects associated with benzodiazepine use are cognitive deficits, described clinically as anterograde amnesia. While relatively little data are available regarding the effects of selective efficacy compounds on cognitive function, a study by Soto et al. (2013, Neuropsychopharmacology 38:2315-25) evaluated the effects of TPA023B on cognition in rhesus monkeys, in comparison with the conventional benzodiazepine triazolam. In a delayed matching-to-sample (DMTS) task of visual recognition memory and self-ordered spatial search (SOSS) task of spatial working memory, triazolam significantly reduced accuracy in both procedures, consistent with cognitive impairing effects reported with conventional benzodiazepines. In contrast, TPA023B did not alter accuracy or the number of trials completed in either task, suggesting that this compound lacks effects on visual/spatial memory.

MRK-898 was not pursued into clinical development due to business reasons (Atack et al. Cur Topics Med Chem 2011, 11:1176-1202). MRK-898 has equal binding affinity at α1, α2, α3, and α5 GABA_{A} subtypes but differs in terms of intrinsic efficacy at these subtypes, with higher positive modulation at α2 and α3 vs. α1 and α5 subtypes. MRK-898 exhibits effective pharmacokinetics in multiple species, with half-life values and clearance suitable for once/day dosing in humans. Predicted toxicology was conducted by the National Institute on Drug Abuse, Division of Therapeutics and Medical Consequences. In vitro tests for cardiovascular (Comprehensive in Vitro Proarrhythmia Assay, CIPA) and mutagenicity (Ames Test) were negative. In CIPA, hERG channel binding occurred at doses estimated to be 64,000-fold higher than therapeutic doses in rat. No significant off-target sites of action were identified.

In some embodiments, a compound for treating benzodiazepine misuse and/or use disorder is according to Formula I:

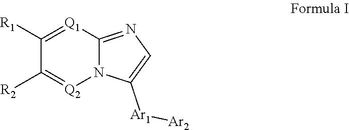

In Formula I, R₁ and R₂ are independently H, Cl, F, CF₃, CN, alkyl (e.g., C1-C5), alkoxy (e.g., C1-C5), OCF₃, or an isopropanol group:

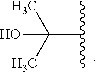 [SMILES: *C(C)(C)O]

In certain preferred embodiments, one of R₁ and R₂ is H, and the other is CH₃, CF₃, or an isopropanol group. For example, in some embodiments, R₁ is CH₃, CF₃, or an isopropanol group and R₂ is H.

Q₁ and Q₂ are each independently C or N. In preferred embodiments, one or both of Q₁ or Q₂ are N, giving the compound an imidazopyrimidine or imidazotriazine core.

Ar₁ and Ar₂ are each 6-membered aromatic rings, each of which are optionally substituted with one or two substituents. Each of Ar₁ and Ar₂ may independently include one or more heteroatoms selected from O or N.

In some embodiments, Ar₁ and Ar₂ are each independently a phenyl, pyridine, pyridazine, pyrimidine, or pyrazine. In embodiments where one or both of Ar₁ or Ar₂ are substituted, the substituents comprise one or more of Cl, F, CF₃, CN, alkyl (e.g., C1-C5), alkoxy (e.g., C1-C5), or OCF₃. In preferred embodiments, the substituents are F and/or CN.

In some embodiments, Ar₁ and Ar₂ form a biphenyl group:

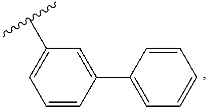 [SMILES: *c1cccc(-c2ccccc2)c1]

wherein one or both of the phenyl groups are optionally substituted F and/or CN.

In some embodiments, compounds of Formula I may be fully or partially deuterated.

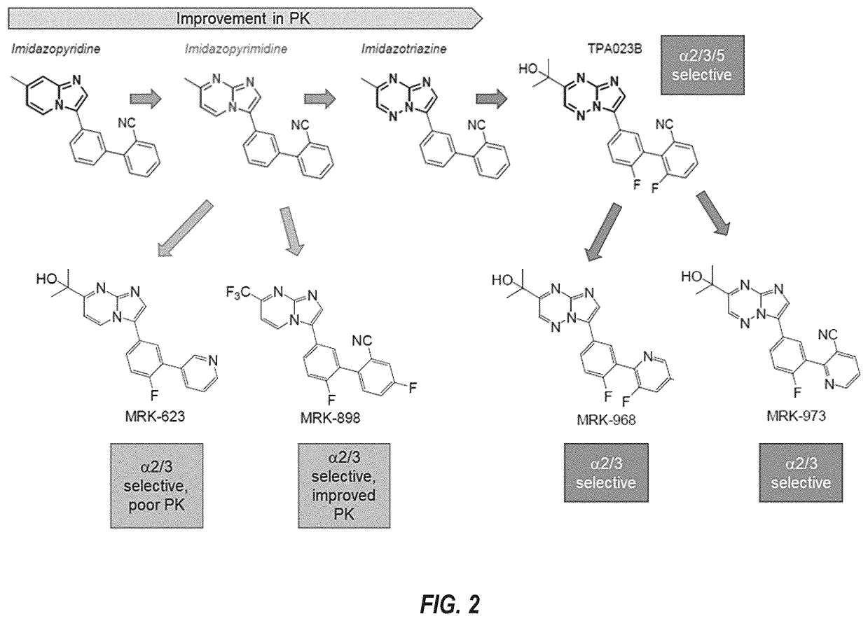

FIG. 2 illustrates a few exemplary compounds according to Formula I. Sequential addition of nitrogen atoms to the benzimidazole core of imidazopyridine enhances bioavailability and half-life without affecting binding and efficacy characteristics (Maramai et al., 2020), resulting in imidazopyrimidines and imidazotriazines as key analogues. TPA023B, a preferred compound for the embodiments described herein, is an imidazotriazine with a fluorinated biphenyl structure. MRK-968 and MRK-973 are notable variants of TPA023B. Analogues of the key intermediary, imidazopyrimidine, have improved plasma half-lives by fluorination, with MRK-898, for example, having effective half-life, bioavailability, and selectivity (rhesus monkey bioavailability of 49%, half-life of 13 h; antagonist at α1/α5GABA_{A} subtypes; Atack, 2011). MRK-623 of this series has fair bioavailability but a very short half-life (0.5 h in rhesus monkey). However, a deuterated formulation of such compounds can improve half-life.

In some embodiments, a compound for treating benzodiazepine misuse and/or use disorder includes a triazolopyridazine core according to Formula II:

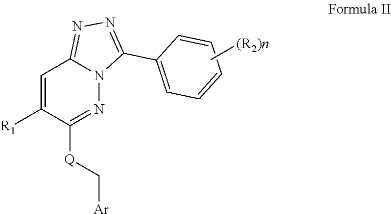

In Formula II, R₁ is H, Cl, F, CF₃, CN, alkyl (e.g., C1-C5), or cycloalkyl (e.g., cyclopropyl, cyclobutyl, cyclopentyl). In some preferred embodiments, R₁ is isopropyl or cyclobutyl.

R₂ is H, Cl, F, CF₃, CN, alkyl (e.g., C1-C5), alkoxy (e.g., C1-C5), or OCF₃, and n is 1 or 2. In some preferred embodiments, R₂ is F.

Q is C, N, O, S, sulfone, or sulfoxide. In some preferred embodiments, Q is O.

Ar is an optionally substituted diazole, triazole, or pyridine. In some preferred embodiments, Ar is a substituted triazole:

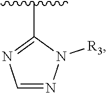

wherein R₃ is H or C1-C2 alkyl.

FIG. 3 illustrates a few exemplary compounds according to Formula II. The triazolopyridazines include TPA023, which was advanced to Phase II clinical trials but halted due to cataract development. Post hoc analyses showed significant anxiolysis in patients with Generalized Anxiety Disorder in three clinical trials (Atack 2009, Adv Pharmacol 57:137-85). As shown, replacement of the bicyclic group of the starting triazolopyridazine molecule increases efficacy. The poor PK of MRK-067 is remedied by fluorination of the phenyl group. Replacement of the bicyclic group of triazolopyridazine with t-butyl substitutions provides L-838,417 and TPA023, both with no efficacy at α1GABA_{A} subtypes. Poor PK has been addressed by the development of deuterated L-838,417 (also referred to as “C 21191” or “CTP-354”).

In some embodiments, a compound for treating benzodiazepine misuse and/or use disorder includes a pyridazine core according to Formula III:

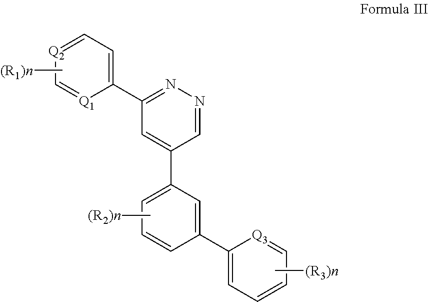

In Formula III, each of Q₁, Q₂, and Q₃ are independently C or N. In some preferred embodiments, at least one of Q₁ or Q₂ is N and Q₃ is N.

Each R₁ (n is 1 or 2) is H, Cl, F, CF₃, CN, C1-C5 alkyl, C1-C5 alkoxy, or OCF₃. Likewise, each R₂ (n is 1 or 2) is H, Cl, F, CF₃, CN, C1-C5 alkyl, C1-C5 alkoxy, or OCF₃ and each R₃ (n is 1 or 2) is H, Cl, F, CF₃, CN, C1-C5 alkyl, C1-C5 alkoxy, or OCF₃. In some preferred embodiments, R₁, R₂, and R₃ are F.

FIG. 4 illustrates a few exemplary compounds of Formula III. These compounds were originally developed as α5GABA_{A}-selective ligands, but instead had efficacy profiles as partial positive modulators with selectivity when fluorination approaches were used to address metabolic lability. Initial PK of all three compounds is relatively good, with the possible exception of MRK-547 in which in vitro results were not correlated with in vivo findings (Atack, 2011). The three compounds have differing selectivity, with MRK-100 showing α3GABA_{A} partial modulation only. This is noteworthy because a compound with full positive modulation of α3GABA_{A} receptors can have relatively low abuse potential (Meng et al. 2020; J Psychopharmacol 34:348-357).

### Treatment of Benzodiazepine Misuse and/or Use Disorder

The compounds described herein may be effectively utilized for treating benzodiazepine misuse and/or use disorder. An effective dose of such a compound, when administered to a subject in need thereof, beneficially provides an anxiolytic effect as a result of its partial modulator function, but with positive reinforcing effects less than those from benzodiazepines (including from “conventional benzodiazepines”) so as not to transfer abuse of one compound to another.

The compounds described herein also beneficially provide antagonist effects and function to suppress the effects of benzodiazepines (including “conventional benzodiazepines”). As demonstrated herein, the suppressive effect is not merely a side effect of sedative and/or motor effects of the compound but is instead directly related to antagonism of the GABA_{A} receptor. As further demonstrated herein, in at least some circumstances the suppression of benzodiazepine effects is insurmountable. That is, the suppression cannot be completely countered by simply increasing the dose of benzodiazepines.

In some embodiments, the compound is administered at a dose of about 0.005 mg/kg to about 10 mg/kg, or more preferably at a dose of about 0.01 mg/kg to about 1 mg/kg. For example, compounds described herein may be administered at doses of about 0.01, 0.02, 0.03, 0.05, 0.06, 0.07, 0.08, 0.09, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mg/kg, or within a dosage range with any two of the foregoing values as endpoints. Preferred dosages for humans may be about 0.5 mg/70 kg (0.007 mg/kg) to about 5.0 mg/70 kg (0.07 mg/kg), or about 1.0 mg/70 kg (0.01 mg/kg) to about 2.0 mg/70 kg (0.03 mg/kg). Higher doses are also possible but are not believed to be efficient use of the compound given that up to about 90-95% of benzodiazepine binding sites are occupied at doses as low as about 0.3 mg/kg to 1.0 mg/kg (e.g., 0.32 mg/kg i.v. provides 95% or greater occupancy in baboons; oral doses to Sprague Dawley rats provides 0.3 mg/kg=87%, 1.0 mg/kg=96%, and 3.0 mg/kg=99% occupancy).

Beneficially, compounds described herein are capable of providing both anxiolytic effects (as a result of partial modulator functionality) and benzodiazepine suppression effects (as a result of antagonist functionality) simultaneously. These effects enable the compounds to be effectively used to treat benzodiazepine misuse and/or use disorder.

As demonstrated herein, the beneficial effects of administration of the disclosed compounds are achievable without inducing substantial motor coordination deficits or appetite suppression in the subject. As further demonstrated herein, the beneficial effects are achievable without substantially affecting breathing frequency, tidal volume, or minute volume in the subject.

Moreover, compounds described herein enable partial modulator and antagonist effects without precipitating benzodiazepine withdrawal. This beneficially enables treatment of subjects that are physically dependent on benzodiazepines without the need for (or at least with significantly less need for) prior or concurrent detoxification treatment, which can be difficult and time-consuming.

### Pharmaceutical Compositions

While it is possible for the compounds described herein to be administered alone, it may be preferable to formulate the compounds as pharmaceutical compositions (e.g., formulations). A subject will most often be a human, but this disclosure also covers situations where compounds are administered to mammals (e.g., as part of a testing protocol).

The compositions may include injectable suspensions, solutions, sprays, lyophilized powders, syrups, elixirs, and the like. Any suitable dosage form may be used. For example, a composition of the disclosure, having a desired degree of purity, is mixed with one or more pharmaceutically acceptable carriers and/or excipients.

As used herein the terms “pharmaceutically acceptable” and “physiologically acceptable” mean a biologically compatible formulation, gaseous, liquid, or solid, or mixture thereof, which is suitable for one or more routes of administration, in vivo delivery, or contact. A formulation is compatible where it does not induce adverse side effects that outweigh any prophylactic or therapeutic effect or benefit.

In an embodiment, the pharmaceutical compositions may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH and may range from a pH of about 3 to a pH of about 11, preferably about pH 3 to about pH 7, depending on the formulation and route of administration. In alternative embodiments, it may be preferred that the pH is adjusted to a range from about pH 5 to about pH 8.

Formulations for parenteral or oral administration can be solids, liquid solutions, emulsions, or suspensions. An exemplary pharmaceutical composition may be formulated as a lyophilized solid that is reconstituted with a physiologically compatible solvent or carrier prior to administration.

Acceptable carriers, excipients, or stabilizers are nontoxic to recipients at the dosages and concentrations employed, and include, but are not limited to, water, saline, phosphate buffered saline, dextrose, glycerol, ethanol, or combinations thereof, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN® PLURONICS® or polyethylene glycol (PEG).

The non-ionic surfactant can include medium chain mono-, di-, and triglycerides (i.e., glycerol esters of 1-3 C8-C10 fatty acids) and esters (mono- and/or di-) of PEG and medium chain fatty acids. An example includes the commercially available lipid compound Labrasol ALF® (“Labrasol”), which is a non-ionic surfactant formed from alcoholysis between polyethylene PEG-8 and medium chain triglycerides (primarily including C8-C10 fatty acids). Labrasol includes: mono-, di- and triglycerides (<10 wt. %); PEG-8 monoesters (40-55 wt. %); PEG-8 diesters (10-20 wt. %), free (non-esterified) PEG-8 (20-30 wt. %), and free glycerol (<5 wt. %).

Suitable excipients may be carrier molecules that include large, slowly metabolized macromolecules such as proteins, polysaccharides, polylactic acids, polyglycolic acids, polymeric amino acids, and amino acid copolymers. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol, and ethanol; wetting or emulsifying agents; pH buffering substances; and the like. Liposomes are also included within the definition of pharmaceutically acceptable excipients.

Pharmaceutically acceptable excipients particularly suitable for use in conjunction with tablets include, for example, inert diluents, such as celluloses, calcium or sodium carbonate, lactose, calcium, or sodium phosphate; disintegrating agents, such as cross-linked povidone, maize starch, or alginic acid; binding agents, such as povidone, starch, gelatin, or acacia; and lubricating agents, such as magnesium stearate, stearic acid, or talc. Pharmaceutical compositions may also be formulated as dispersible powders and granules suitable for preparation of a suspension by the addition of suitable excipients.

Pharmaceutical compositions may be formulated as suspensions comprising a compound as disclosed herein in admixture with at least one pharmaceutically acceptable excipient suitable for the manufacture of a suspension. Excipients suitable for use in connection with suspensions include suspending agents, such as sodium carboxymethylcellulose, methylcellulose, hydroxypropyl methylcellulose, sodium alginate, polyvinylpyrrolidone, gum tragacanth, gum acacia, dispersing, or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); polysaccharides and polysaccharide-like compounds (e.g., dextran sulfate); glycoaminoglycans and glycosaminoglycan-like compounds (e.g., hyaluronic acid); and thickening agents, such as carbomer, beeswax, hard paraffin, or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

Controlled release preparations can be produced by the use of polymers to complex or absorb the compounds. A controlled-release formulation can be prepared using appropriate macromolecules (for example, polyesters, polyamino acids, polyvinyl, pyrrolidone, ethylenevinylacetate, methylcellulose, carboxymethylcellulose, or protamine sulfate) known to provide the desired controlled release characteristics or release profile. Another possible method to control the duration of action by a controlled-release preparation is to incorporate the active ingredients into particles of a polymeric material such as, for example, polyesters, polyamino acids, hydrogels, polylactic acid, polyglycolic acid, copolymers of these acids, or ethylene vinylacetate copolymers.

Additionally, or alternatively, microencapsulation of pharmaceuticals can give a controlled release. The reproducibility of polymer synthesis and the microencapsulation process, the cost of the microencapsulation materials and process, the toxicological profile, the requirements for variable release kinetics and the physicochemical compatibility of the polymer and the compounds are all factors that can be considered in selecting a suitable polymer. Examples of useful polymers are polycarbonates, polyesters, polyurethanes, polyorthoesters, and polyamides, including those that are biodegradable. Microcapsules can be prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nanoparticles and nanocapsules) or in macroemulsions. Such techniques are disclosed in New Trends and Developments in Vaccines, Voller et al. (eds.), University Park Press, Baltimore, Md., 1978 and Remington's Pharmaceutical Sciences, 16th edition.

The carrier can include poly (d, 1-lactide-co-glycolide) (PLGA). A body of data has accumulated on the adaption of PLGA for the controlled release of compounds, for example, as reviewed by Eldridge, J. H., et al., Current Topics in Microbiology and Immunology. 1989, 146:59-66. The PLGA microencapsulation process uses a phase separation of a water-in-oil emulsion. In an example, the compound of interest is prepared as an aqueous solution and the PLGA is dissolved in suitable organic solvents such as methylene chloride and ethyl acetate. These two immiscible solutions are co-emulsified by high-speed stirring. A non-solvent for the polymer is then added, causing precipitation of the polymer around the aqueous droplets to form embryonic microcapsules. The microcapsules are collected and stabilized with one of an assortment of agents (e.g., polyvinyl alcohol (PVA), gelatin, alginates, polyvinylpyrrolidone (PVP), methyl cellulose), and the solvent removed by either drying in vacuo or by solvent extraction.

The pharmaceutical compositions may be in the form of oil-in water emulsions. The oil-in-water emulsion can be based, for example, on light liquid paraffin oil (European Pharmacopea type); isoprenoid oil such as squalane, squalene, EICOSANE™ or tetratetracontane; oil resulting from the oligomerization of alkene(s), e.g., isobutene or decene; esters of acids or of alcohols containing a linear alkyl group, such as plant oils, ethyl oleate, propylene glycol di (caprylate/caprate), glyceryl tri (caprylate/caprate) or propylene glycol dioleate; esters of branched fatty acids or alcohols, e.g., isostearic acid esters. The oil can be used in combination with emulsifiers to form the emulsion. The emulsifiers can be nonionic surfactants, such as esters of sorbitan, mannide (e.g., anhydromannitol oleate), glycerol, polyglycerol, propylene glycol, and oleic, isostearic, ricinoleic, or hydroxystearic acid, which are optionally ethoxylated, and polyoxypropylene-polyoxyethylene copolymer blocks, such as the Pluronic® products, e.g., L121. The adjuvant can be a mixture of emulsifier(s), micelle-forming agent, and oil such as that which is commercially available under the name Provax® (IDEC Pharmaceuticals, San Diego, Calif.). The emulsion may also contain sweetening and flavoring agents. Syrups and elixirs may be formulated with sweetening agents, such as glycerol, sorbitol, or sucrose. Such formulations may also contain a demulcent, a preservative, a flavoring, or a coloring agent.

Additionally, the pharmaceutical compositions may be in the form of a sterile injectable preparation, such as a sterile injectable aqueous emulsion or oleaginous suspension. This emulsion or suspension may be formulated according to the known art using those suitable dispersing or wetting agents and suspending agents which have been disclosed herein. The sterile injectable preparation may also be a sterile injectable solution or suspension in a non-toxic parenterally acceptable diluent or solvent, such as a solution in 1,2-propane-diol. The sterile injectable preparation may also be prepared as a lyophilized powder. Among the acceptable vehicles and solvents that may be employed are water, Ringer's solution, and an isotonic sodium chloride solution. In addition, sterile fixed oils may be employed as a solvent or suspending medium. For this purpose, any bland fixed oil may be employed including synthetic mono- or diglycerides. In addition, fatty acids such as oleic acid may be used in the preparation of injectables.

In some embodiments, aqueous solubility enhancers such as cyclodextrins may be included. Example cyclodextrins include hydroxypropyl, hydroxyethyl, glucosyl, maltosyl and maltotriosyl derivatives of cyclodextrin. An exemplary cyclodextrin solubility enhancer is hydroxypropyl-o-cyclodextrin (BPBC).

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, but are not limited to, mineral salts (e.g., AlK(SO₄)₂, AlNa(SO₄)₂, AlNH(SO₄)₂, silica, alum, Al(OH)₃, Ca₃(PO₄)₂, kaolin, or carbon).

Pharmaceutical compositions can be prepared by associating the active ingredient with liquid carriers or finely divided solid carriers or both, and then, if necessary, shaping the product. For example, a tablet may be made by compression or molding. Compressed tablets may be prepared by compressing, in a suitable machine, an active ingredient in a free-flowing form such as a powder or granules, optionally mixed with a binder, lubricant, inert diluent, preservative, surface-active or dispersing agent. Molded tablets may be produced by molding, in a suitable apparatus, a mixture of powdered compound moistened with an inert liquid diluent. The tablets may optionally be coated or scored and may be formulated so as to provide a slow or controlled release of the active ingredient therein.

Pharmaceutical compositions can be packaged in unit dosage forms for ease of administration and uniformity of dosage. A “unit dosage form” refers to a physically discrete unit suited as unitary dosages for the subject to be treated; each unit containing a predetermined quantity of compound optionally in association with a pharmaceutical carrier which, when administered in one or more doses, is calculated to produce a desired effect (e.g., prophylactic or therapeutic effect or benefit). Unit dosage forms can contain a daily, weekly, or monthly dose, or an appropriate fraction thereof, of an administered compound. Unit dosage forms also include, for example, capsules, troches, cachets, lozenges, tablets, ampules, and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. The individual unit dosage forms can be included in multi-dose kits or containers. Pharmaceutical formulations can be packaged in single or multiple unit dosage forms for ease of administration and uniformity of dosage.

The compositions disclosed herein can be administered at any frequency and as a single bolus or multiple doses, for as long as appropriate. Exemplary frequencies are typically from 1-5 times, 1-3 times, 2-times, or once monthly. In some embodiments, the compounds will be administered for a period of continuous therapy, for example for a week or more, or for months or years.

The disclosed compounds can be administered in a base form and/or in the form of one or more salts. A presently preferred salt is a fumarate salt, which for certain compound can be included in a hemifumarate stoichiometric ratio. One such example is the hemifumarate salt of MRK-898 shown below:

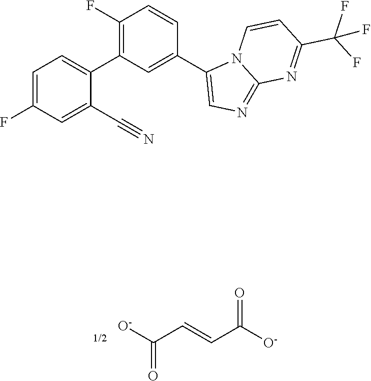 [SMILES: N#Cc1cc(F)ccc1-c1cc(-c2cnc3nc(C(F)(F)F)ccn23)ccc1F ; O=C([O-])/C=C/C(=O)[O-]]

Such a salt form can exhibit 0.25% (w/w) or less, 0.20% (w/w) or less, or 0.15% (w/w) or less water uptake when measured between dryness ant 90% relative humidity (RH) at 25° C. Such a salt form can exhibit physical stability and chemical stability for at least 1 week when measured in accelerated stability testing at 40° C. and 75% RH. As used herein in the context of salt forms, “physical stability” means less than 1% (w/w) change in the solid form of the composition, and “chemical stability” means less than 0.5% (w/w) increase in impurities (i.e., compounds different from the salt form and any intended carriers/excipients) in the composition.

### EXAMPLES

### Example 1: Conflict Model of Anxiolysis

The conflict procedure methodology includes a two-component multiple fixed-ratio, fixed-ratio plus punishment schedule (mult FR, FR+PUN). An important feature of this model is that it is highly predictive of clinically active doses in human subjects (Rowlett et al. 2006, Psychopharmacology 184:201-11). One component of this schedule was associated with a distinctive visual stimulus (e.g., red light) and the other component was associated with a different stimulus (e.g., green light). In each component, completion of 10 responses (FR 10) produced a food pellet followed by a brief (10 sec) timeout period. The component ended following delivery of 5 food pellets or 5 min, whichever occurred first. A 10-min timeout period occurred before each of four cycles with two components (1 red, 1 green) per cycle. When performance in both components of the multiple schedule was stable, responding in one component was suppressed by superimposing a FR schedule of response-produced electric shock. Under the superimposed schedule, every nth response (n=15−25, adjusted for each monkey depending on individual performance) produced shock. Once responding was stable (at least three sessions in which suppressed responding was less than 50% of non-suppressed responding, with no upward or downward trend in response rates in either component), investigational compounds were administered i.v. during the extended timeout periods using a cumulative dosing technique. This technique allowed determination of a four-point dose-response function in a single session.

Table 2 summarizes data for TPA023B in the conflict procedure (n=4 monkeys, 2 females and 2 males), with alprazolam as a positive control. Average responses when vehicle was administered was 2.2 in the non-suppressed component, decreased by ˜98% by shock presentation in the suppressed component (mean=0.05 responses). For non-suppressed responding, TPA023B had no effects on mean rates of responding or percent of control over a 100-fold dose range (0.01 to 1.0 mg/kg, i.v.). The comparator drug, alprazolam, markedly attenuated non-suppressed responding at a dose of 0.3 mg/kg, i.v. For suppressed responding, TPA023B significantly enhanced rates of suppressed responding, reaching non-suppressed levels of responding at 0.1 mg/kg, i.v., i.e., this compound showed an anti-conflict effect. This effect was similar to that observed with 0.03 mg/kg, i.v., of alprazolam. A striking characteristic of TPA023B in this procedure is the lack of attenuation of rates of responding at higher doses. In contrast, rate-suppressing effects are shown here with alprazolam and demonstrated with a series of other conventional benzodiazepines (Rowlett et al. 2006).

TABLE 2
Effects of TPA023B on suppressed and non-suppressed
responding in a rhesus monkey conflict procedure.

| Treatment (mg/kg, i.v.) | Non-Suppressed | Suppressed |
|---|---|---|
| | Mean responses/s (SEM) | |
| Vehicle | 2.2 (0.16) | 0.05 (0.01) |
| 0.01 | 2.0 (0.08) | 0.03 (0.02) |
| 0.1 | 2.0 (0.15) | 2.46 (0.40)* |
| 1.0 | 2.1 (0.97) | 2.00 (0.75)* |
| Alprazolam (0.03) | 1.8 (0.31) | 1.9 (0.10)* |
| Alprazolam (0.3) | 0.18 (0.09)* | 0.10 (0.10) |
| | % Non-Suppressed Vehicle (SEM) | |
| Vehicle | 100 (7.5) | 2.4 (0.13) |
| 0.01 | 95 (3.7) | 1.4 (0.82) |
| 0.1 | 95 (7.2) | 112 (16)* |
| 1.0 | 96 (46) | 91 (37)* |
| Alprazolam (0.03) | 82 (18) | 86 (4.5)* |
| Alprazolam (0.3) | 8.2 (4.0)* | 4.5 (4.5) |

*P < 0.05 vs. corresponding vehicle control, Bonferroni t-tests;
N = 4 monkeys

In summary, TPA023B demonstrated anti-conflict effects to a degree associated with conventional anxiolytic benzodiazepines, but unlike benzodiazepines, it showed no propensity to impair the subjects' ability to complete responses, i.e., to induce motor coordination deficits.

### Example 2: Observation/Sedation

For the data shown here, the behavior of each monkey (n=4 total) was scored using a focal animal approach as described previously (Duke et al. 2018, J Pharmacol Exp Ther 366:145-57). Briefly, a trained observer blind to the drug treatments observed a specific monkey for 5 min and recorded each instance that a particular behavior occurred during 15-s intervals. Scores for each behavior were calculated as the number of 15-s bins in which the behavior occurred (e.g., a maximum score would be 20). For sedation measures, structured exposure to stimuli were included in the observation sessions (Duke et al., 2018). When a monkey was observed to have closed eyes for more than 3 s, an assessment of the animal's responsiveness to the stimuli was determined. Specifically, the observer presented three stimuli: 1) walked at a normal pace towards the cage, 2) spoke the animal's name, and 3) moved the lock used to secure the door of the cage. If the monkey responded immediately (i.e., opened eyes and oriented to the observer), “rest/sleep posture” was scored. If the monkey attended more slowly (i.e., >3 s following stimuli), the observer scored “moderate sedation”. If the monkey did not open eyes across/throughout the 15-s interval after all three stimuli, the observer noted the loss of ability to respond to external stimuli and made as assessment of “deep sedation”.

When scoring moderate or deep sedation, the monkey might be observed to be assuming an atypical posture that differed from the characteristic rest/sleep posture (e.g., unable to keep an upright posture). The assessment of sedation was initiated during the 5-min sampling period if the animal presented, at any time during that period, with its eyes closed for longer than 3 s. The result of this assessment was recorded for each remaining 15-sec interval of the 60-sec epoch unless eyes opened. Afterwards, eyes closing again initiated the assessment. If eyes remained closed, then the assessment was repeated at the beginning of the next 60-sec epoch15-sec interval. Therefore, the maximum score for sedation during a 5-min period was 20 (four possible scores for 4×15-sec intervals per min, 5 min total; i.e., 4 possible scores×5 min=20). The order in which animals were observed and the observer performing the scoring each day was randomized. Four observers participated in the scoring throughout the duration of the study; each observer underwent a minimum of 20 hours of training and met an inter-observer reliability criterion of ≥90% agreement with all other observers.

The five-min sampling periods were repeated at multiple times following i.v. injection of TPA023B or vehicle (minutes post injection): 5, 10, 20, 40, 80, 160, 320. Scores were cumulated across the time periods, resulting in a maximum possible cumulative score of 140. TPA023B was compared with alprazolam, and the results have been published by Duke et al. (2018). The key results are summarized in Table 3. Notably, however, Duke et al. 2018 did not discuss the potential use of TPA023B as a treatment for benzodiazepine misuse and/or use disorder.

TABLE 3
Summary of significant behavioral effects
(out of 22 total) following multiple doses
of alprazolam and TPA023B.
Significant effects were identified as at least one dose
significantly different from vehicle (N = 4 monkeys).

| Compounds | Increase | Decrease |
|---|---|---|
| Alprazolam | Rest/Sleep Posture, Moderate | Passive Visual, Tactile/Oral |
| | Sedation, Deep Sedation, | Exploration |
| | Observable Ataxia | |
| TPA023B | Rest/Sleep Posture | Tactile/Oral Exploration |

See above for definitions of Rest/Sleep Posture, Moderate and Deep Sedation.
For other definitions:
Observable ataxia—any slip, trip, fall, loss of balance;
Passive Visual—animal is standing or sitting motionless with eyes open;
Tactile/Oral Exploration—any tactile or oral manipulation of the cage or environment.

Consistent with findings from studies with human patients (Atack et al. 2010), TPA023B was well-tolerated by rhesus monkeys up to doses of 1.0 mg/kg, i.v. (30-fold higher than the lowest dose inducing rest/sleep posture). At doses of 0.03 to 1.0 mg/kg, i.v., TPA023B induced rest/sleep posture that corresponds to a mild form of sedation in which the subject can be roused easily by stimuli.

### Example 3: Self-Administration

The abuse potential of novel compounds can be determined by use of i.v. self-administration procedures, in which a compound is available to a subject previously trained to press a lever to obtain a known drug of abuse. Our standard procedure is to use a progressive-ratio (PR) schedule, in which an increasing number of responses are required to obtain an injection. The last response requirement completed is referred to as a “break point” (BP) and represents the maximum amount of “work” a subject will perform to obtain the drug or compound.

In the present example, eight rhesus monkeys (4 females, 4 males) were trained to respond under a schedule of i.v. midazolam (a conventional benzodiazepine) injection at 0.03 mg/kg/injection. Once self-administration had been acquired (>20 injections/daily 2-hour sessions), monkeys were trained under the PR schedule as described by Shinday et al. (2013). Daily experimental sessions consisted of five components, each made up of four trials (i.e., 20 trials total). Each trial within a component had the same response requirement and was separated by a timeout (30 min of no lights or lever availability) in order to minimize drug accumulation due to repeated injections in a single session. A session ended when the response requirement was not met within a 30-min limited hold for two consecutive trials or when all 20 trials are completed. The PR sequence had an initial response requirement of 40 per injection and doubled at the start of each component (i.e., response requirements of 40, 80, 160, 320, 640). Training sessions alternated with drug and saline according to the following 5-session cycles: DSSDD, SDDSS (S=saline or D=drug). Responding under the PR schedule was considered stable across the cycles when the number of injections/session were less than 5 for at least three sessions of saline availability and greater than 11 for at least three sessions of drug availability, with no consistent upward or downward trends. Test sessions were inserted into the sequence every third session, e.g., DSTSDTD, STDDTSS, and so on (T=test). Test conditions included TPA023B, in comparison with the conventional benzodiazepine lorazepam, which has a similar duration of action to the test compound.

A range of doses for lorazepam (0.001-0.03 mg/kg/injection) and TPA023B (0.003-0.03 mg/kg/injection) was tested and for both, 2+ doses maintained a significantly higher level of mean number of injections/session than maintained by vehicle alone (vehicle generally resulted in 3-4 injections/session, since no cues signaled drug availability). In order to directly compare lorazepam and TPA023B self-administration, we here analyzed BP values at the doses that maintained the highest level of mean injections/session and compared these BP values with those obtained after vehicle tests.

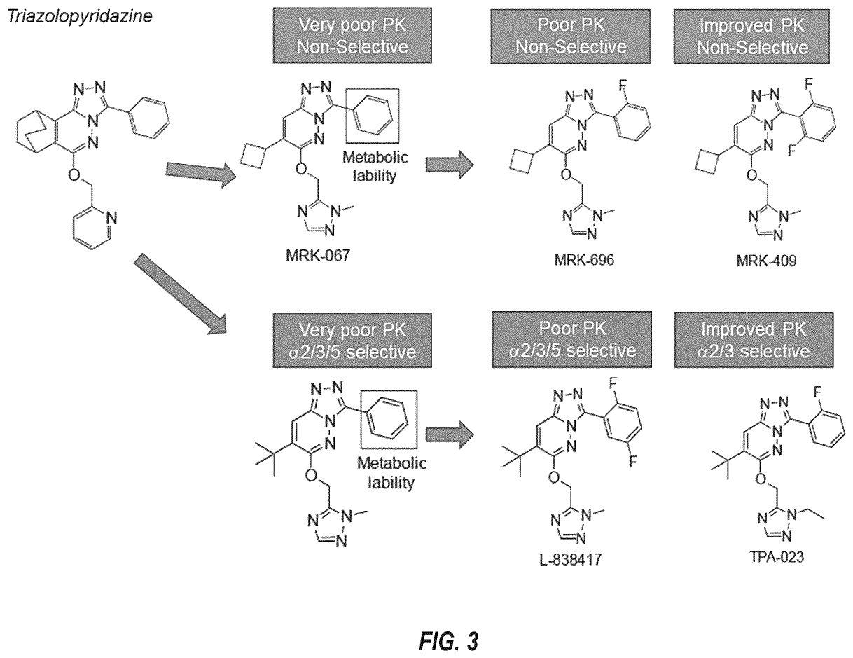

As can be seen in FIG. 5, lorazepam at the dose in which self-administration was highest resulted in a median BP of 320, whereas the highest response requirement maintained by TPA023B was 160. These values were significantly different (Dunnett's test for ranks, p<0.05). Both lorazepam and TPA023B, as expected, maintained median BP values significantly higher than those maintained by vehicle (Dunnett's tests for ranks, p<0.05). These findings suggest that while TPA023B was a reinforcer, it had significantly lower reinforcing effectiveness than the conventional benzodiazepine lorazepam. These findings are consistent with TPA023B being predominantly a partial positive modulator/antagonist at GABA_{A} receptor subtypes.

### Example 4: Specific Suppression of Self-Administration of Benzodiazepines

TPA023B showed anti-conflict effects, consistent with this compound having anxiolytic-like effects, similar to conventional benzodiazepines. When sedative-motor effects were compared with the conventional benzodiazepine alprazolam (Xanax®), TPA023B showed only mild sedation and no motor deficits. And when evaluated for abuse potential, TPA023B demonstrated a lower potential for abuse than lorazepam (Ativan®), which is self-administered to the same degree as all conventional benzodiazepines in our PR procedure. A key question for evaluation of this compound as a potential pharmacotherapy is the extent to which it attenuates the reinforcing effects of a conventional benzodiazepine, under conditions in which TPA023B is a weak reinforcer.

To test this, we have trained monkeys to self-administer the short-acting conventional benzodiazepine midazolam (0.056 mg/kg/injection, i.v.) in the PR procedure, as described above. During test sessions, we initially kept the training dose of midazolam constant, and administered i.v. pretreatments of TPA023B (0.003, 0.01, 0.03, 0.1, 0.3 mg/kg, of vehicle) immediately prior to the beginning of the session. A key consideration for any pharmacotherapy, as well as interpretation of blockade of reinforcing effects generally, is the extent to which the blockade is specific to the drug target and not a generalized suppression of behavior, e.g., due to sedative-motor effects inhibiting the ability of the monkey to press the lever. Another possible confounding factor that would potentially result in blockade of drug taking is general sickness or perceptual alterations. In particular, appetite suppression would represent an undesirable side effect in a benzodiazepine addiction pharmacotherapy.

To evaluate the extent to which suppression of lever-pressing behavior by TPA023B represents non-specific inhibition, we also tested food self-administration under the PR schedule. For these experiments, test sessions were scheduled similar to those with midazolam dose/vehicle, except that 2 food pellets (1-g BioServ flavored food pellets) were available as reinforcers rather than midazolam dose. All other aspects of the test was the same, including response requirements and TPA023B pretreatment conditions.

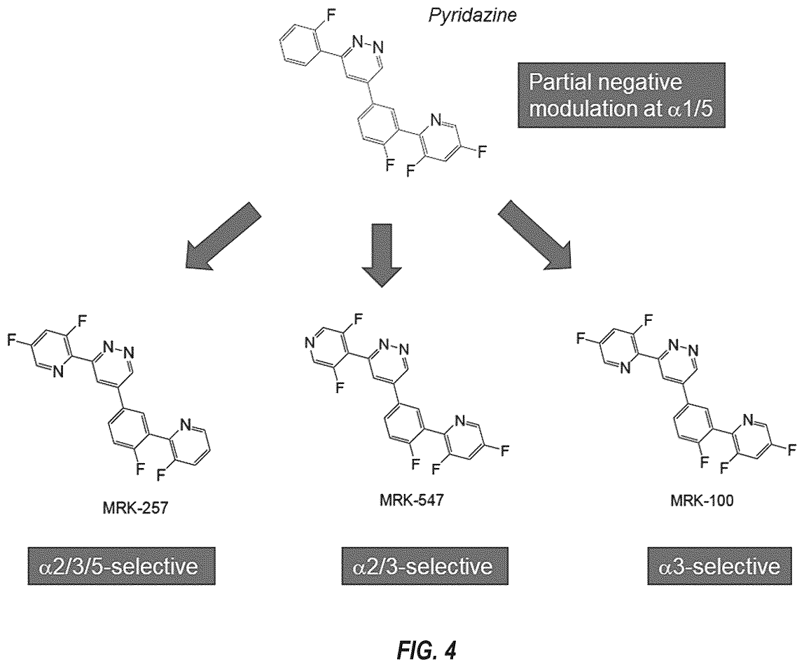

FIG. 6 shows the effects of pretreatments with TPA023B on self-administration of two doses of midazolam and food pellets in four rhesus monkeys. Points above “V” (vehicle) show that both midazolam and food pellet delivery maintained between 11 and 15 reinforcers per session out of 20 possible, which was essentially the same as baseline responding. Increasing doses of TPA023B reduced midazolam-maintained responding at both doses, with a >50% reduction at 0.03 mg/kg. In contrast, TPA023B did not alter food-maintained responding up to 0.3 mg/kg. These findings clearly show that TPA023B blocks the self-administration of the benzodiazepine midazolam without altering food self-administration, showing the desired specificity and suggesting that the suppression of responding is not due to sedative-motor effects and/or attenuation of general reinforcing processes.

### Example 5: Insurmountable Suppression of BZ Self-Administration Following TPA023B

To further evaluate the extent to which attenuation of responding was due to pharmacological antagonism and to evaluate an exemplary range of effective doses, we evaluated single doses of TPA023B administered prior to sessions in which differing doses of midazolam were tested.

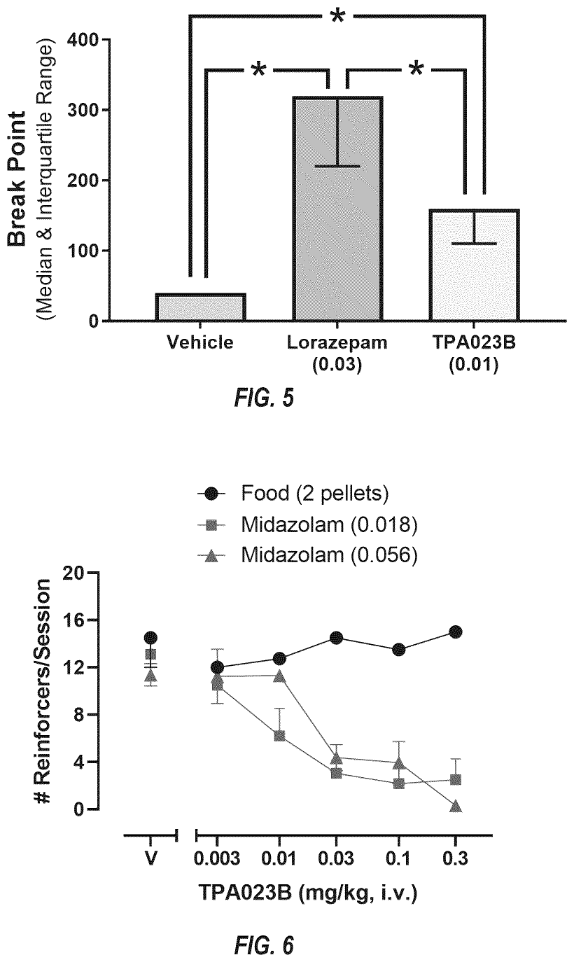

FIGS. 7A and 7B show both raw data (FIG. 7A, number of injections/session) and percent maximum (FIG. 7B, % of each monkey's maximum number of injections/session). The Figures demonstrate that midazolam alone resulted in dose-dependent self-administration of 0.018, 0.056, and 0.18 mg/kg/injection, but not 0.0056 mg/kg/injection, with the 0.018 mg/kg/injection dose showing maximal self-administration. Pretreatment with 0.003 mg/kg TPA023B did not attenuate self-administration of midazolam but increasing the TPA023B dose to 0.01 mg/kg shifted the midazolam dose-response function to the right. That is, TPA023B suppressed self-administration of midazolam at 0.018 mg/kg/injection but when the midazolam dose was increased, responding returned to maximal levels (see FIG. 4, bottom panel).

At higher doses of TPA023B, the midazolam dose-response function was shifted to the right and down, with self-administration generally not reaching 50% of maximum, even at the relatively higher midazolam doses. In other words, the suppression of midazolam self-administration by TPA023B was not fully surmountable, meaning that re-establishing midazolam taking cannot occur simply by taking higher doses of the benzodiazepine. These data further support TPA023B's ability to block the effects of a conventional benzodiazepine irrespective of sedative and/or motor effects and demonstrate a surprising insurmountable profile.

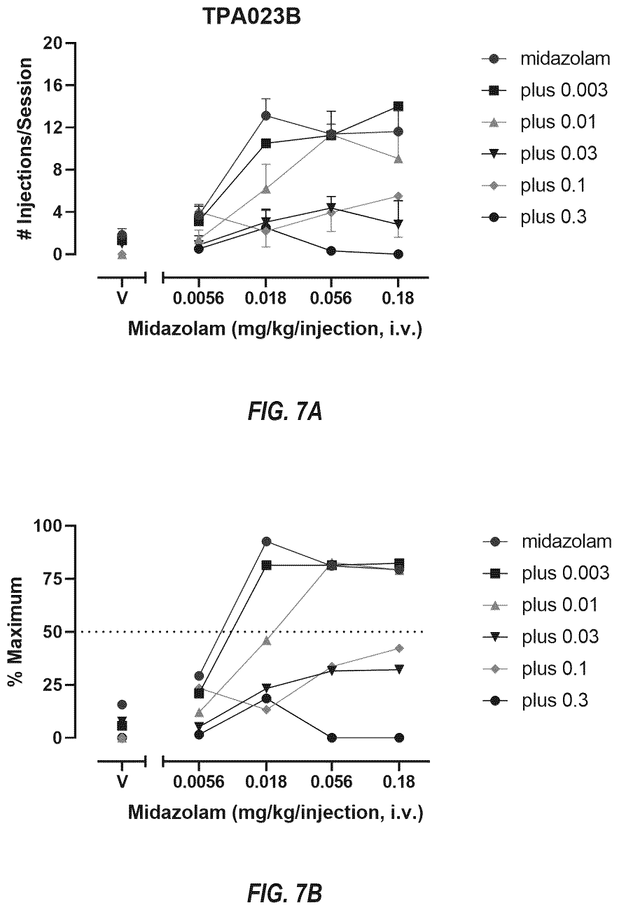

To illustrate the relationship of anxiolysis to blockade of benzodiazepine self-administration, FIG. 8 plots the highest three doses of midazolam self-administration along with the conflict data from Table 2 as average % maximum effect. When plotted as percent maximum, TPA023B increased suppressed responding (“anti-conflict effect”) in a dose-dependent manner. As noted above, TPA023B dose-dependently reduced midazolam self-administration to below 50% for all midazolam doses, with attenuation consistently below 25% for the highest TPA023B dose. For both conflict and blockade of self-administration, TPA023B doses at and above 0.03 mg/kg had both anti-conflict effects and blocked midazolam self-administration. TPA023B thus attenuated the reinforcing effects of midazolam at the same doses that engendered an increase in anxiolytic-like effects, suggesting that TPA023B retains anxiolysis with the ability to block benzodiazepine self-administration.

### Example 6: Evaluation of Respiratory Depression

Although considered safe relative to several other drugs in psychiatric medicine, benzodiazepines are known respiratory depressants. The effects of benzodiazepines on pulmonary ventilation are considered modest; however they do represent risk for aspiration and their use is contraindicated with sleep apnea. Importantly, increased risk of overdose death occurs when benzodiazepines are combined with alcohol and/or opioids. Therefore, we have initiated studies to assess potential respiratory depressant effects of our partial positive modulators.

We first assessed the effects of TPA023B, in comparison with alprazolam, using whole body plethysmography (WBP; DSI systems). For these studies, adult male Sprague-Dawley rats (n=8) were habituated to the chambers for 30 min. The chamber was then filled with 21% oxygen, sealed, and pressure fluctuation measured for 1 min. Next, the chamber was opened and the rats were injected with alprazolam or TPA023B, i.p., and placed into the sealed chamber for a 15 min pretreatment time. The chamber was again filled with 21% oxygen, and for 1 min, fluctuating pressure amounts were recorded to obtain tidal volume (lung capacity), breaths/minute, and minute volume (lung capacity per unit time).

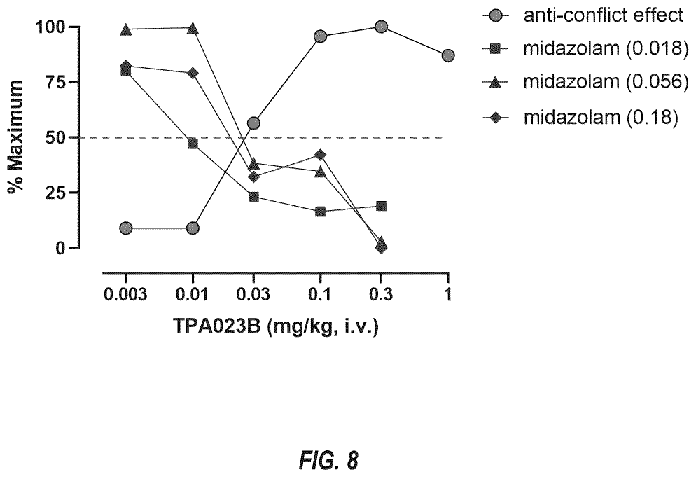

FIG. 9 shows results from testing with alprazolam and TPA023B in 8 rats, tested once or twice per week. Alprazolam significantly reduced breathing frequency and minute volume at 3.0 mg/kg, but not tidal volume. In contrast, TPA023B did not alter any ventilatory parameter over the doses tested. Importantly, the doses were chosen as those that occupied up to 95% of benzodiazepine binding sites (1.0 and 3.0 mg/kg, Atack et al. 2010). These findings demonstrate low toxicity for partial positive modulators, a key property given that benzodiazepine addiction patients are likely to abuse other substances as well.

### Example 7: Evaluation of Physical Dependence & Withdrawal

A well-documented consequence of benzodiazepine exposure, and important contributor to the abuse of these drugs, is physical dependence (Licata & Rowlett 2008, Pharmacol Biochem Behav 90:74-89). Physical dependence occurs when exposure to a drug leads to aversive, or even life-threatening, consequences upon cessation of the drug exposure. Cessation can result either by “spontaneous withdrawal” (abrupt removal of drug treatment) or precipitated withdrawal (administration of a drug with antagonist properties). Benzodiazepine withdrawal signs include gastrointestinal disturbances (e.g., nausea, vomiting), anxiety, sleeplessness, tremors, and in severe cases, seizures. Alleviation of withdrawal may be a major factor contributing to relapse to benzodiazepine taking.

Mild withdrawal syndromes can occur even after a single, relatively large bolus exposure to a benzodiazepine. This effect may be an earlier contributor to benzodiazepine addiction, particularly in vulnerable patients (e.g., induction of mild anxiety conceivably could drive an anxiety patient to seek additional benzodiazepines). Acute dependence can be demonstrated in non-human animals, providing a means to study early events in the addiction cycle but also providing an approach that reduces risk to the subjects.

Acute dependence can occur after single exposures to the benzodiazepine chlordiazepoxide, but not to an α2/3GABA_{A} selective ligand (Fischer et al. 2013, Psychopharmacology 227:347-54). Here, the extent to which TPA023B can precipitate withdrawal in rats following a single, large bolus dose of diazepam was studied. For these studies, adult Sprague-Dawley rats (N=8) were trained to press a lever 10 times to receive a food pellet (FR 10 schedule) during daily 10 minute sessions. Once rates of responding were stable, doses of diazepam, the non-selective benzodiazepine antagonist flumazenil, and TPA023B were administered prior to the session.

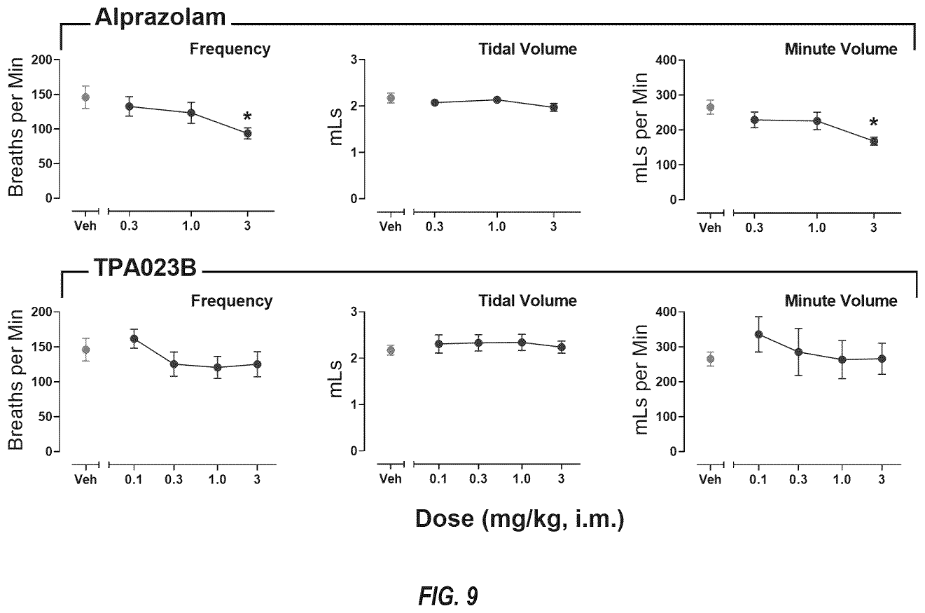

FIGS. 10A to 10C show that mean rates of responding (expressed as percent of baseline control) were significantly decreased by diazepam, but not flumazenil or TPA023B.

We next varied the time between injection and testing for diazepam at 3.0 mg/kg, and found the most robust effects at 30 min pre-treatments before the session, and no effects at 60 min pre-treatment.

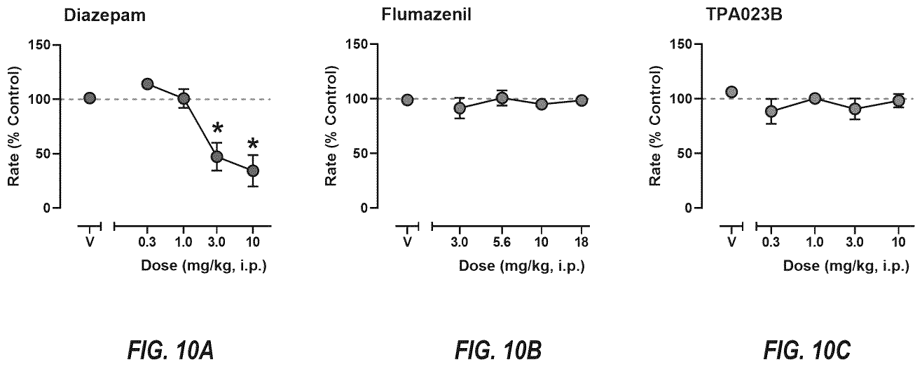

To assess the extent to which flumazenil and TPA023B precipitate withdrawal, we evaluated both compounds using the 60-min pre-treatment time, in which diazepam's effects had dissipated. As shown in FIG. 11A, re-testing the doses of flumazenil robustly suppressed responding at all doses tested compared with the rates after flumazenil alone. That is, flumazenil disrupted lever-pressing behavior 60 min after a bolus injection of diazepam, indicative of a withdrawal state.

In contrast, as shown in FIG. 11B, TPA023B did not alter rates of responding at any dose tested (doses shown to occupy up to ˜95% of benzodiazepine binding sites in brain (3.0 and 10 mg/kg)). These findings illustrate that TPA023B and similar compounds are not expected to precipitate withdrawal in dependent subjects, which would be a significant leap forward in treatment initiation, since patients with benzodiazepine addiction would not need to be detoxified from the benzodiazepine prior to therapy.

### Example 8: Representative Imidazopyrimidine and Triazolopyridazine Compounds

TPA023B includes an imidazotriazine core. The compounds MRK-623 and L-838,417 were also evaluated as exemplary compounds with imidazopyrimidine and triazolopyridazine cores, respectively.

The triazolopyradizine L-838,417 has functional selectivity for α2/3/5GABA_{A} receptors and is an α1GABA_{A} antagonist. This profile is very similar to that of TPA023B despite the structural differences. The imidazopyrimidine, MRK-623, which has a different selectivity profile from TPA023B in that it is α2/3GABA_{A} subtype selective. For both compounds, we chose 3.0 mg/kg, i.p., in rats based on published literature or pilot studies in our laboratories.

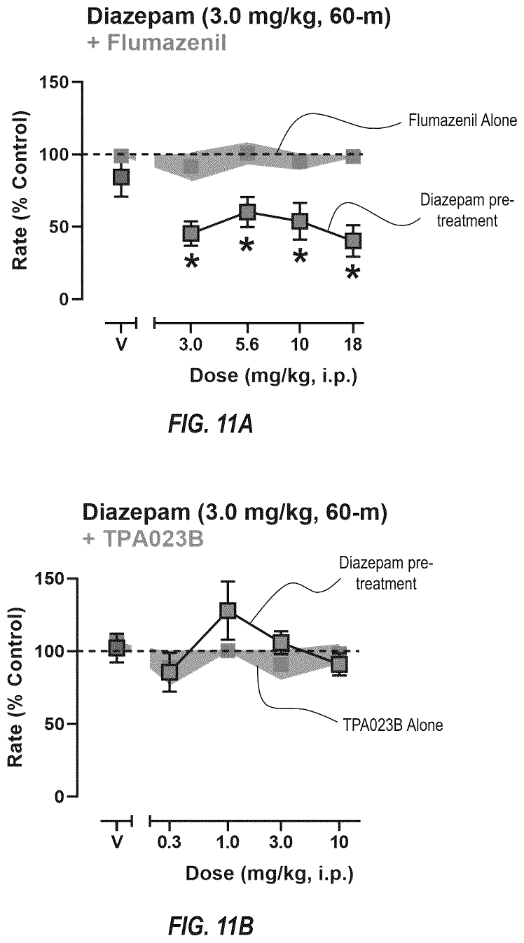

FIGS. 12A and 12B illustrate the results of acute dependency testing using these compounds. As shown, the dose of 3.0 mg/kg for the non-selective antagonist flumazenil, as well as L-838,417 and MRK-623, blocked the ability of diazepam (30 min pretreatment; FIG. 12A) to suppress rates of responding. When these same doses of the ligands were administered to rats after a 60-min pretreatment of diazepam (when diazepam's effects had dissipated; FIG. 12B), flumazenil induced a significant decrease in rates of responding, consistent with a withdrawal-like state and acute dependence. However, neither L-838,417 nor MRK-623 altered rates of responding, indicating that these compounds, like TPA023B, do not induce acute physical dependence.

### Example 9: Reversal of Withdrawal Induced by GABA_{A} Receptor Antagonist

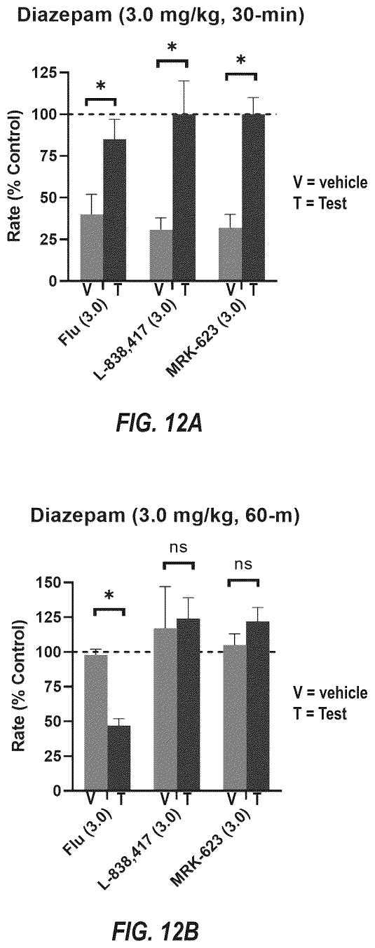

FIG. 13 illustrates results of testing on rhesus monkeys, demonstrating that TPA023B lacks withdrawal-precipitating effects and can reverse withdrawal induced by the non-selective GABA_{A} antagonist flumazenil. As shown, mean rates of responding for food (expressed as percent of baseline control) were significantly reduced by administering flumazenil (a competitive antagonist that is not selective for any subtype) after midazolam (see bottom panel, third column from the left), indicating that flumazenil acutely precipitates withdrawal symptoms. Administering TPA023B after midazolam did not significantly affect response rate (bottom panel, second column from the left). Moreover, when both TPA023B and flumazenil were administered after midazolam, TPA023B blocked the withdrawal-promoting effects of flumazenil and allowed the monkeys to return to baseline feeding response rates (see bottom panel, fourth column from the left). These findings demonstrate that TPA023B can attenuate or remove withdrawal symptoms in individuals physically dependent on benzodiazepine drugs.

### Example 10: Insurmountable Suppression of BZ Self-Administration Following MRK-898

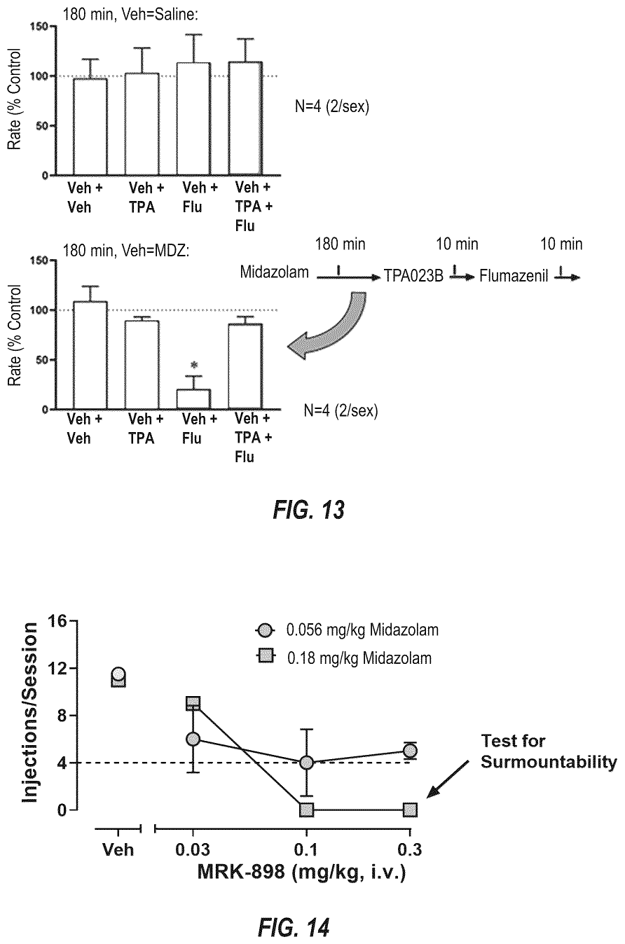

We trained rhesus monkeys to press levers to obtain i.v. injections of the standard BZ, midazolam. As shown in FIG. 14 (Veh=midazolam at doses shown), a dose of 0.056to 0.18 mg/kg/injection of midazolam typically maintains about 11-12 injections/session. A session consists of availability of 20 injections in a 10-hour daily period. When saline is available, monkeys typically self-administer about 4 injections/session (dashed line), representing a comparative baseline. When i.v. injections of MRK-898 (at the doses shown on the horizontal axis) were administered 5 min before the session, self-administration of 0.056mg/kg/injection of midazolam was reduced to saline-like levels at doses of 0.03 to 0.3 mg/kg of MRK-898. To determine whether increasing the dose of midazolam can surmount the blockade induced by MRK-898, we evaluated the effects of pre-treatment of 0.03 to 0.3 mg/kg of MRK-898 with a higher dose of 0.18 mg/kg/injection of midazolam. As shown, increasing the midazolam dose did not result in an increase in self-administration, consistent with insurmountable blockade. In summary, MRK-898 blocked BZ self-administration, an effect that could not be surmounted by increasing the BZ dose. This is a beneficial property for an effective BZ misuse/use disorder treatment.

### Example 11: MRK-898 Does Not Promote Sedation

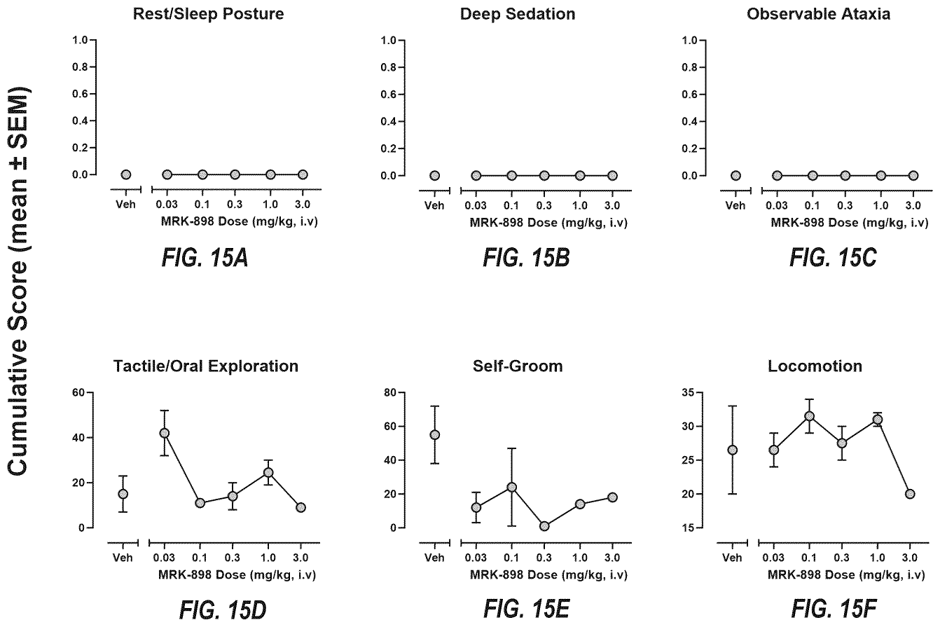

This study was conducted using a system that objectively measures sedative-motor effects, based on anesthesiology standards, in rhesus monkeys (Duke et al. J Pharmacol Exp Ther 2018, 366:145-157). Trained observers used a time-sampling approach to record the frequency of behaviors associated with sedative-motor effects, as well as ongoing species-typical behavior, following i.v. administration of drugs/test compounds. Using this approach, we demonstrated that MRK-898 lacks sedative-motor effects over a 100-fold dose range, including doses that blocked midazolam self-administration (FIGS. 15A-15C). This lack of sedative effects is surprising because similar compounds induce at least mild sedation (rest/sleep posture) at relatively low doses. This is the first partial and selective α2/α3 GABA_{A} positive modulator shown to lack sedative-motor effects in our observation procedures. FIGS. 15D-15F show species-typical behavior, showing moderate or absent effects.

### Example 12: MRK-898 Blocks Sedation Caused by BZ Administration

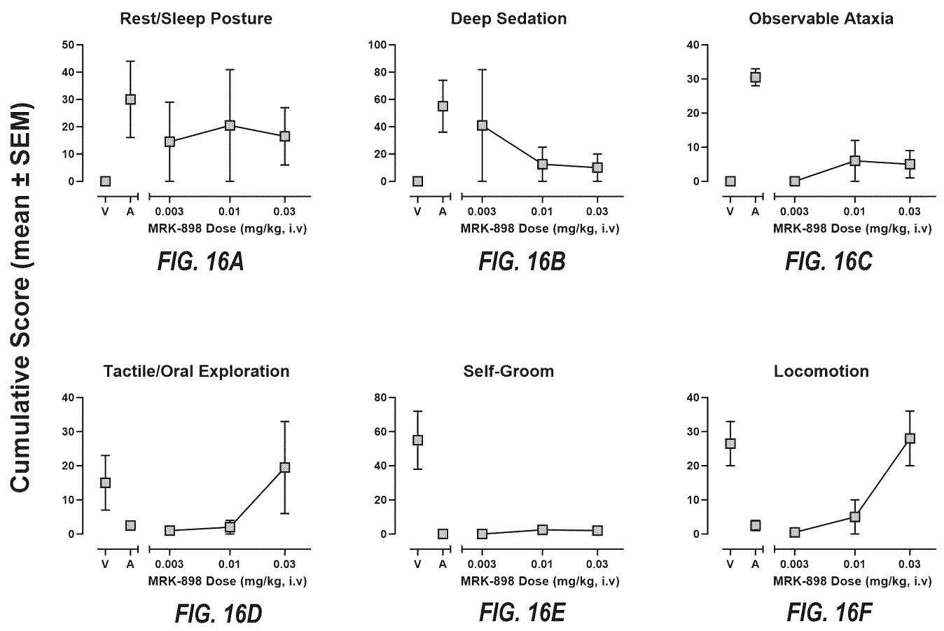

As shown in FIGS. 16A-16F, MRK-898 also blocked the sedative-motor effects of a standard BZ, alprazolam. In this study, alprazolam was administered i.v. at a dose (0.56 mg/kg) that induced rest/sleep posture, deep sedation, and observable ataxia compared with vehicle (FIGS. 16A-16C, compare “A” vs. “V”). When administered after alprazolam, MRK-898 reduced these sedative-motor effects, with minimum doses of 0.003 mg/kg and 0.01 mg/kg being effective to block observable ataxia and sedation, respectively. MRK-898 also reversed the suppression of species-typical behaviors, except for self-groom (FIGS. 16D-16F). These findings demonstrate that MRK-898 is likely occupying receptors at i.v. doses of 0.003 mg/kg and above, and that it can robustly block the sedative-motor effects of a standard BZ.

### Example 13: Oral Administration of MRK-898

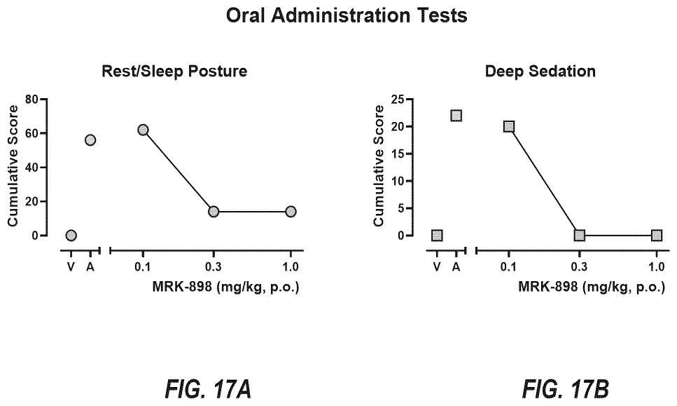

MRK-898 has extremely low aqueous solubility (˜0.007 mg/ml) that is not altered by adjusting pH to physiologically relevant levels. Nevertheless, when the studies of Example 12 were repeated with a 3-hour pretreatment, orally administered MRK-898 completely blocked the sedative effects of 0.56 mg/kg alprazolam administered i.v., 5 min before the session (FIGS. 17A and 17B). MRK-898 was approximately 3-fold less potent oral vs. i.v., indicating a reasonable degree of oral bioavailability. MRK-898 was therefore orally active in rhesus monkeys, indicating that a successful formulation for this compound for oral use in humans is highly likely.

### Example 14 (Prophetic): MRK-898 Physical Dependence Studies

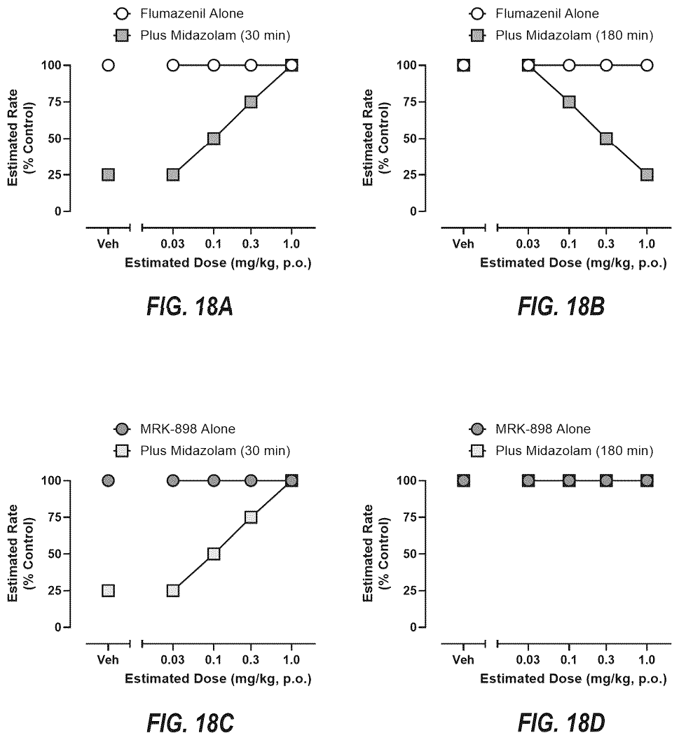

As demonstrated in Examples 7 and 9, a compound with functional selectivity for α2, α3, and a5 GABA_{A} subtypes (TPA023B) did not precipitate acute withdrawal in rats or rhesus monkeys. This was demonstrated in a procedure in which subjects pressed response levers to obtain food pellets. When a BZ (e.g., midazolam) is administered prior to a lever-pressing session, relatively high doses of the BZ will suppress responding below levels observed after vehicle administration. The non-selective BZ antagonist, flumazenil, will block these rate suppressing effects in a dose-dependent manner, as shown in the example of FIG. 18A (midazolam administered 30 minutes before testing, flumazenil dose shown on horizontal axis). When the BZ is administered several hours before the lever-pressing session (e.g., 180 minutes), then as the drug clears the body, rates of responding return to normal. Subsequent administration of flumazenil will suppress rates of responding (presumably due to the animals being in a withdrawal state), as shown in the example of FIG. 18B (midazolam administered 180 minutes before testing, flumazenil dose shown on horizontal axis).

A similar (prophetic) study is carried out using MRK-898. As shown in FIG. 18C, the MRK-898 is predicted to act as an antagonist of BZs administered in proximity (e.g., 30 minutes prior) to the session, similar to flumazenil. However, unlike flumazenil, after the longer BZ pretreatment time (e.g., 180 minutes), MRK-898 is predicted to minimize or avoid precipitation of withdrawal effects, as shown in FIG. 18D. The results are predicted to demonstrate that MRK-898 can be safely administered to BZ-dependent individuals.

### Example 15: MRK-898 Salt Formulation

Solid-state characterization of MRK-898 in its base form revealed that the compound is a crystalline solid, anhydrous, and has a melting point at 183.7° C. The material is non-hygroscopic with less than 0.15% (w/w) moisture uptake between 0-90% relative humidity (RH) at 25° C. and showed no physical form conversion in response to humidity change.

Primary salt screen was performed using 19 acids (salt formers) in 6 solvents. Among potential salts, hemifumarate, monobesylate, and monomaleate were selected for scale-up study based on criteria of crystalline form, molecular weight, melting point, and thermal profile. At a larger scale of 350 mg free base, salt formations of hemifumarate and monobesylate demonstrated reproducibility and high yields, at 93.2% and 78.9% respectively. Both hemifumarate and monobesylate were non-hygroscopic with approximately 0.15% (w/w) water uptake between dryness and 90% RH at 25° C. In addition, both showed no solid form change in response to humidity. Scale up of maleate salt was not robust and reproducible.

Stability studies showed that MRK-898 hemifumarate salt was physically and chemically stable at 40° C./75% RH for 1 week. The solid form did not change, and purity remained at 100% according to HPLC-UV analysis. For the monobesylate salt, the same storage condition did not change the solid form of the compound, but impurities grew from 0.89% (w/w) to 1.69% (w/w) after 1 week.

The hemifumarate and monobesylate salts presented low solubility (0.007 mg/mL equivalent of free base) in Fasting State Simulated Gastric Fluid (FaSSGF), pH 1.5, and Fasting State Simulated Intestinal Fluid (FaSSIF), pH 6.5. While both salts remained the same solid form, monobesylate exhibited significantly reduced crystallinity in biorelevant media.

Although enhanced solubility was not obtained, the hemifumarate salt exhibited effective stability, thermal characteristics, and melting point. Given the ability of MRK-898 to provide effects even with relatively low bioavailability (see Example 13), the hemifumarate salt may be effective despite low solubility.

### Example 16: MRK-898 Lipid Formulation

The commercially available lipid compound Labrasol ALF® (“Labrasol”) is a self-emulsifying non-ionic surfactant formed from alcoholysis between polyethylene PEG-8 and medium chain triglycerides (primarily including C8-C10 fatty acids). Labrasol includes: mono-, di- and triglycerides (<10 wt. %); PEG-8 monoesters (40-55 wt. %); PEG-8 diesters (10-20 wt. %), free PEG-8 (20-30 wt. %), and free glycerol (<5 wt. %).

A series of excipients were tested based on an estimated maximum clinical dose of 10 mg. MRK-898 was added at >10 mg/mL in 12 different excipients, and after 4 hours, a further excess of compound was added to those excipients where MRK-898 visually appeared to be dissolved. The test excipients containing MRK-898 were kept at 40° C. overnight. Samples where MRK-898 appeared to be solubilized were filter centrifuged using 0.22 μm nylon filters and filtrate was analyzed by HPLC-UV. Of the tested excipients, Labrasol solubilized MRK-898 at >21.4 mg/ml, and was one of two excipients retaining high purity (>98%).

MRK-898 exhibited low solubility (0.003 mg/mL) in FaSSGF, pH 1.5, and FaSSIF, pH 6.5. However, the relatively high solubility of MRK-898 in Labrasol, combined with relatively high potency in vivo (see, e.g., Example 13), indicates that MRK-898/Labrasol can be effectively formulated in an oral dosage form such as a gel-cap. The Labrasol can also function as a permeability enhancer (PE) to increase MRK-898 intestinal bioavailablity.

### Exemplary Features

Embodiments of the present disclosure may include, but are not necessarily limited to, features recited in the following clauses:
Clause 1. A method of treating benzodiazepine misuse and/or use disorder, the method comprising: administering to a subject having benzodiazepine misuse and/or use disorder an effective amount of a compound comprising the structure:

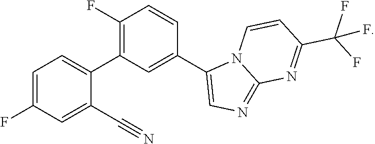 [SMILES: N#Cc1cc(F)ccc1-c1cc(-c2cnc3nc(C(F)(F)F)ccn23)ccc1F]

Clause 2. The method of clause 1, wherein administration of the compound suppresses benzodiazepine self-administration in the subject.
Clause 3. The method of clause 2, wherein suppression of benzodiazepine self-administration is insurmountable and is not overcome by increased benzodiazepine dose.
Clause 4. The method of clause 2 or clause 3, wherein suppression of benzodiazepine self-administration is independent of sedative or motor effects of the compound.
Clause 5. The method of any preceding clause, wherein administration or the compound does not induce sedative effects in the subject.
Clause 6. The method of any preceding clause, wherein administration or the compound does not significantly affect motor coordination in the subject.
Clause 7. The method of any preceding clause, wherein administration of the compound blocks sedative effects of a benzodiazepine.
Clause 8. The method of any preceding clause, wherein administration of the compound blocks ataxia caused by a benzodiazepine.
Clause 9. The method of any preceding clause, wherein the compound is administered parenterally at a dose of at least 0.003 mg/kg, optionally up to 1-3 mg/kg.
Clause 10. The method of any preceding clause, wherein the compound is administered enterally at a dose of greater than 0.1 mg/kg, optionally up to 1-3 mg/kg.
Clause 11. The method of clause 10, wherein the compound is administered orally.
Clause 12. The method of clause 11, wherein the compound is administered orally in a gelcap dosage form.
Clause 13. The method of any preceding clause, wherein administration of the compound does not precipitate benzodiazepine withdrawal.
Clause 14. The method of any preceding clause, wherein the compound is administered in the form of a composition including a non-ionic surfactant.
Clause 15. The method of clause 14, wherein the non-ionic surfactant comprises: mono-, di-, and/or triglycerides of a C8-C10 fatty acid; and/or mono-and/or diesters of PEG and a C8-C10 fatty acid.
Clause 16. The method of clause 15, wherein the non-ionic surfactant further comprises free PEG.
Clause 17. The method of any preceding clause, wherein the compound comprises a fumarate salt.
Clause 18. The method of clause 17, wherein the fumarate salt is a hemifumarate salt.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

When the terms “about,” “approximately,” “substantially,” or the like are used in conjunction with a stated amount, value, or condition, it may be taken to mean an amount, value or condition that deviates by less than 10%, less than 5%, less than 1%, less than 0.1%, or less than 0.01% of the stated amount, value, or condition.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent may also include two or more such referents.

It will also be appreciated that embodiments described herein may also include properties and/or features described in one or more separate embodiments and are not necessarily limited strictly to the features expressly described for that particular embodiment. Accordingly, the various features of a given embodiment can be combined with and/or incorporated into other embodiments of the present disclosure.

1. A method of treating benzodiazepine misuse and/or use disorder, the method comprising:
administering to a subject having benzodiazepine misuse and/or use disorder an effective amount of a compound comprising the structure:

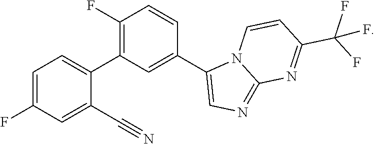 [SMILES: N#Cc1cc(F)ccc1-c1cc(-c2cnc3nc(C(F)(F)F)ccn23)ccc1F]

2. The method of claim 1, wherein administration of the compound suppresses benzodiazepine self-administration in the subject.

3. The method of claim 2, wherein suppression of benzodiazepine self-administration is insurmountable and is not overcome by increased benzodiazepine dose.

4. The method of claim 2, wherein suppression of benzodiazepine self-administration is independent of sedative or motor effects of the compound.

5. The method of claim 1, wherein administration or the compound does not induce sedative effects in the subject.

6. The method of claim 1, wherein administration or the compound does not significantly affect motor coordination in the subject.

7. The method of claim 1, wherein administration of the compound blocks sedative effects of a benzodiazepine.

8. The method of claim 1, wherein administration of the compound blocks ataxia caused by a benzodiazepine.

9. The method of claim 1, wherein the compound is administered parenterally at a dose of at least 0.003 mg/kg, optionally up to 1-3 mg/kg.

10. The method of claim 1, wherein the compound is administered enterally at a dose of greater than 0.1 mg/kg, optionally up to 1-3 mg/kg.

11. The method of claim 10, wherein the compound is administered orally.

12. The method of claim 11, wherein the compound is administered orally in a gelcap dosage form.

13. The method of claim 1, wherein administration of the compound does not precipitate benzodiazepine withdrawal.

14. The method of claim 1, wherein the compound is administered in the form of a composition including a non-ionic surfactant.

15. The method of claim 14, wherein the non-ionic surfactant comprises:
mono-, di-, and/or triglycerides of a C8-C10 fatty acid; and/or
mono-and/or diesters of PEG and a C8-C10 fatty acid.

16. The method of claim 15, wherein the non-ionic surfactant further comprises free PEG.

17. The method of claim 1, wherein the compound comprises a fumarate salt.

18. The method of claim 17, wherein the fumarate salt is a hemifumarate salt.

19. A composition comprising:
(i) a compound comprising the structure: 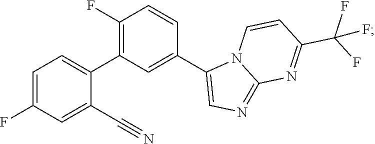 [SMILES: N#Cc1cc(F)ccc1-c1cc(-c2cnc3nc(C(F)(F)F)ccn23)ccc1F]
and
(ii) a non-ionic surfactant comprising:
mono-, di-, and/or triglycerides of a C8-C10 fatty acid; and/or
mono- and/or diesters of PEG and a C8-C10 fatty acid.

20. A hemifumarate salt composition comprising the structure: [chemical structure]
